(19) United States
(12) Patent Application Publication
(10) Pub. No.: US 2024/0157082 A1
(43) Pub. Date: May 16, 2024

(54) EVAPORATOR DEVICE

(71) Applicant: Alveon GmbH, Freienbach (CH)

(72) Inventors: André Geilen, Freienbach (CH); Simon Geiss, Freienbach (CH); Ralf Martin Riess, Freienbach (CH)

(21) Appl. No.: 18/282,436

(22) PCT Filed: Mar. 16, 2022
(86) PCT No.: PCT/EP2022/056786

§ 371 Date: Sep. 15, 2023

(30) Foreign Application Priority Data
Mar. 16, 2021 (DE) 102021202544.7

(51) Int. Cl. A61M 16/14; A61M 15/06

(52) U.S. Cl. CPC A61M 16/147; A61M 15/06; A61M 2205/0211; A61M 2205/3653; A61M 2205/8206

(57) ABSTRACT

An evaporator device for evaporating a substance may include an evaporator, two electrical connections, a blocking conductor, and an electrical current path for an electrical supply of the evaporator. The evaporator may include an electrically conductive evaporator ceramic with a receiving structure that receives the substance during operation. The electrical current path may extend through the two electrical connections, the evaporator ceramic, and the blocking conductor. The evaporator ceramic may be configured to homogeneously provide heat in a thermal operating range between an operation starting temperature and an operation end temperature. The blocking conductor may be heat-transmittingly connected to the evaporator ceramic. The blocking conductor may configured such that, upon exceeding the operation end temperature, an electrical resistance of the at least one blocking conductor abruptly increases.

### CROSS-REFERENCE TO RELATED APPLICATIONS

This application claims priority to International Patent Application No. PCT/EP2022/056786, filed on Mar. 16, 2022, and German Patent Application No. DE 10 2021 202 544.7, filed on Mar. 16, 2021, the contents of both of which are hereby incorporated by reference in their entirety.

### TECHNICAL FIELD

The present invention relates to an evaporator device for evaporating a substance, which comprises an evaporator and electrical connections for the electrical supply of the evaporator. Further, the invention relates to an inhaler having such an evaporator device.

### BACKGROUND

An evaporator device generally serves for evaporating a liquid and is usually employed in inhalers. Usually, such an evaporator device comprises a wick, which saturates itself with the liquid to be evaporated, and a heating wire, which is connected to the wick in a heat-transmitting manner and upon electrical supply, generates heat in order to evaporate the liquid stored in the wick.

From DE 10 2018 119 566 A1 an evaporator device having an evaporator of an electrically conductive porous element is known, which at the same time is employed for storing the liquid and for heating, for the purpose of evaporating the liquid, in order to avoid the formation of dry hits, i.e. locally excessive heating, which can result in the destruction of the wick.

From DE 10 2016 120 803 A1 and evaporator device having an evaporator consisting of a doped and electrically conductive evaporator ceramic is known. The evaporator ceramic is provided with controlled micro-channels having a predetermined orientation, through which liquid flows for the purpose of evaporation. Upon electrical supply, the evaporator ceramic generates heat in order to evaporate the liquid received therein. Further, the evaporator device comprises a flow control device, which controls the flow of the liquid through the micro-channels, in order to achieve a dosing of the liquid to be evaporated.

From DE 10 2017 123 868 A1 an evaporator device is known, which, like the evaporator device known from DE 10 2016 120 803 A1, comprises an evaporator ceramic with parallel micro-channels for storing and evaporating the liquid. In order to avoid a bubble formation in the inlet region of the micro-channels, a wick structure is additionally provided in the inlet region and, being in areal contact, is connected to the inlet side.

Disadvantageous in evaporator devices known from the prior art is the complicated manufacture and the lacking and/or elaborate control of the evaporation of the liquid to be evaporated.

### SUMMARY

The present invention therefore deals with the object of stating for an evaporator device of the type mentioned above and for an inhaler having such an evaporator device, improved or at least other embodiments, which are characterised in particular by a simplified manufacture and/or improved control of the evaporation.

According to the invention, this object is solved through the subject matter of the independent claim(s). Advantageous embodiments are the subject matter of the dependent claim(s).

The present invention is based on the general idea of providing an evaporator device with an electrically conductive ceramic comprising a receiving structure, wherein the receiving structure serves for receiving a substance to be evaporated, and wherein the electrically conductive ceramic at the same time serves for generating heat for evaporating the substance received in the receiving structure, and providing an electrical conductor for the electrical supply of the electrically conductive ceramic, which is arranged in a path of the electric current for the electrical supply of the electrically conductive ceramic and is connected to the electrically conductive ceramic in a heat-transmitting manner, wherein the conductor, upon exceeding a predetermined temperature, exhibits an abruptly increasing electrical resistance. The use of the receiving structure of the electrically conductive ceramic with simultaneous use of the electrically conductive ceramic as electric heater for evaporating the substance result in a simplified manufacture of the evaporator device and allow the controlled evaporation of substance, in particular of a controlled volume of the substance. The abrupt increase of the electrical resistance of the electrical conducted connected to the electrically conductive ceramic in a heat-transmitting manner upon exceeding the predetermined temperature, further results in that upon reaching the predetermined temperature an electrical supply of the electrically conductive ceramic is interrupted or at least reduced in such a manner that the predetermined temperature predetermines a maximum temperature of the electrically conductive ceramic. In a particularly simple and effective manner, this results in that the electrically conductive ceramic during the operation does not exceed the predetermined temperature so that there is a precisely defined control of the evaporation parameters. Further it is thus possible to operate the evaporator ceramic with a power which, without the blocking conductor, would result in overheating. Because of this it is possible without elaborate control, to bring the evaporator ceramic quickly and with high power up to temperatures in the operating range, and keep it in the operating range.

According to the inventive idea, the evaporator device comprises the evaporator. The evaporator includes the electrically conductive ceramic, which in the following is also referred to as evaporator ceramic. At the same time, the evaporator ceramic serves for receiving and storing the substance to be evaporated and for generating heat for evaporating the substance. The storing of the substance takes place by means of the receiving structure of the evaporator ceramic. The evaporator ceramic thus comprises the receiving structure, in which during the operation the substance to be evaporated is received. For the electrical supply of the evaporator, in particular of the evaporator ceramic, the evaporator device, further, comprises two electrical connections. The path of the electric current, in the following also referred to as current path, runs between the two electrical connections and through the evaporator ceramic. The evaporator ceramic is configured in such a manner that, by means of its electrically conductive characteristic, during the operation, upon electrical supply, homogeneously generates heat in the evaporator ceramic in order to evaporate the substance received in the receiving structure. The evaporator, in particular the evaporator ceramic, is designed for the operation in a thermal operating range which is delimited by a lower operation starting temperature and an upper operation end temperature. In other words, the evaporator ceramic for evaporating the substance, upon electrical supply, generates heat in the operating range and thus between the operation starting temperature and the operation end temperature. The homogeneous heat generation of the evaporator ceramic results in a homogeneous temperature or a homogeneous heat distribution in the volume of the evaporator ceramic and consequently in the receiving structure. This results in an even evaporation of the substance throughout the receiving structure. Further, the evaporator device comprises the abovementioned electrical conductor, which in the following is also referred to as blocking conductor. According to the invention, the blocking conductor is arranged in the current path, so that the electric current during the operation flows through the blocking conductor. In addition, the blocking conductor is configured in such a manner that, at the operation end temperature, it has a abruptly increasing electrical resistance. Because of the heat-transmitting connection of the blocking conductor with the evaporator ceramic, there is consequently an abrupt increase of the electrical resistance in the current path and thus an interruption or at least substantial reduction of the electrical supply of the evaporator ceramic when the operation end temperature is reached. Accordingly, the operation end temperature is at least substantially influenced, preferentially defined by means of the blocking conductor.

The heat-transmitting connection of the blocking conductor with the evaporator ceramic is advantageous in such a manner that the temperature of the blocking conductor at least substantially corresponds to the temperature of the evaporator ceramic.

As mentioned, the evaporator ceramic upon electrical supply homogeneously generates heat by means of its electrical conductivity. In particular, the evaporator ceramic is a heating resistor.

The evaporator ceramic can be an electrically conductive ceramic of any kind, provided it comprises the receiving structure and upon electrical supply, in particular on applying an electrical voltage in a predetermined range, homogeneously generates heat in the operating range.

It is conceivable that the evaporator ceramic per se is electrically conductive. This includes for example ceramics of metal oxides, such as titanium oxides, or metal carbides as well as silicon carbides. Likewise, composite ceramics can be employed which comprise electrically conductive and electrically non-conductive networks of different materials, wherein the conductive networks are practically distributed homogeneously in the ceramic. Examples for such composite ceramics are such having metal oxides of different oxidation stage. Mixed oxide ceramics can also be employed, which are used by mixing different starting materials, wherein during the manufacture of the ceramic, typically during the sintering, a new material is created through chemical reactions. Examples for the starting materials are different metal oxides. Further, doped ceramics can be employed, which by doping become electrically conductive. Obviously, any combinations of the mentioned ceramics can also be employed provided the evaporator ceramic is an electrically conductive ceramic with the receiving structure, which during the operation, homogeneously generates heat.

The evaporator device can be operated both continuously and also discontinuously. The evaporator device is configured accordingly.

During the continuous operation, the evaporator ceramic is continuously supplied with substance for at least a limited duration and at least partly evaporates the said substance. The supply with the substance can be realised through a permanent fluidic connection of the evaporator device, in particular of the evaporator ceramic, with a substance container, in which the substance is stored, so that substance continuously flows into the evaporator ceramic. The evaporation takes place for as long as the evaporator device is electrically supplied and operated in the operating range. The evaporated quantity of the substance can be controlled if required via the operating duration of the evaporator device in the operating range.

During the discontinuous operation, the evaporator ceramic is supplied with a predetermined dose of the substance, which is then evaporated. In particular, the evaporator device, in particular the evaporator ceramic, is not continuously supplied with the substance during the discontinuous operation. Thus, the evaporated quantity can be controlled in particular via the volume of the evaporator ceramic and/or the quantity of the substance received in the evaporator ceramic.

During the discontinuous operation, the evaporator device, in particular the evaporator ceramic, can be supplied with substance after the substance previously received in the evaporator ceramic is at least partly evaporated and/or the evaporator device is not operated in the operating range, in particular is out of operation. Thus, the evaporator device, in particular the evaporator ceramic, is refillable.

During the discontinuous operation it is likewise conceivable to already stock the evaporator device with a dose of the substance, which is evaporated during the operation. Here, the evaporator device can be configured for one-time use, i.e. replaceable. In particular, the evaporator device can be configured in the manner of a tablet.

Obviously, mixed operations of discontinuous and continuous operation are also possible.

The receiving structure is advantageously formed and/or moulded integrally in the evaporator ceramic.

Practically, the receiving structure is homogeneously distributed in the evaporator ceramic.

It is conceivable to introduce the receiving structure into the evaporator ceramic by subsequent working. It is conceivable, in particular, to form for example micro-channels in the evaporator ceramic, which are part of the receiving structure or form the receiving structure.

Preferably, the receiving structure comprises pores in the evaporator ceramic. Particularly preferably, the receiving structure consists of pores, i.e. is a pore structure.

The pores of the evaporator ceramic are advantageously formed during the manufacture of the evaporator ceramic, which can take place for example by means of sintering. In other words, the pores for receiving the substance are advantageously introduced not separately, in particular not subsequently, into the evaporator ceramic. Thus, an intrinsic characteristic of the evaporator ceramic provided by the manufacture is used for storing the substance to be evaporated. This results in a simple and cost-effective manufacture of the evaporator ceramic and thus of the evaporator device.

In addition, through the manufacture of the evaporator ceramic, a total volume of the evaporator ceramic defined by the pores and thus volume of the receivable substance can be varied and predetermined. This results in a further simply configured control of the evaporation parameters.

Basically, the evaporator ceramic can comprise pores of any kind.

It is advantageous when the evaporator ceramic comprises pores with a mean size between 0.05 μm and 50 μm. These mean pore sizes, in the case of a liquid as substance, result in such a ratio between the surface and the volume of the respective pore, so that these have capillary forces, which offset, preferentially outweigh the forces acting, due to gravity and/or due to pressure, on a drop-shaped particle of the liquid received in the volume. This results in that the drop-shaped particles, in the following also referred to as droplets, remain in the pores. Consequently, a draining of the droplets and consequently of the liquid from the evaporator ceramic is prevented or at least substantially reduced. Thus, low-viscosity liquids can also be received and stored in the evaporator ceramic. Thus it is also possible with the evaporator ceramic to receive and store a greater variability of liquids of different viscosity without the liquids draining from the evaporator ceramic. As a consequence, the liquids can be provided more cost-effectively and in a wider spectrum. In particular, active substances received in the liquids can thus be provided in a simpler manner and/or with a more precise dose. Thus, the evaporator ceramic and the associated evaporator device can be more easily employed for a controllable inhalation of the said active substances and thus a controllable and/or predetermined dosing of the active substances. The capillary forces described above result, further, in that the evaporator ceramic upon a hydraulic connection with the liquid to be evaporated, saturates itself with the liquid to be evaporated without further action, such as for example an active pumping of the liquid into the evaporator ceramic. Altogether, separate seals of the evaporator ceramic can thus be omitted or at least reduced and/or devices for actively introducing the liquid into the ceramic, omitted. Thus, both the evaporator ceramic and also an associated evaporator device can be easily and cost-effectively realised. Besides an increase of the possible applications of the evaporator ceramic and of the associated evaporator ceramic, a simplified realisation of the same also takes place.

A further advantage of the said mean pore sizes must be seen in that these result in an enlargement of the area of the droplets that are in contact with the evaporator ceramic. In other words, an enlarged area of the evaporator ceramic transmits heat to the droplets for evaporating the liquid. This results in a more even evaporation of the liquid and thus an improved control over the evaporation. In addition, there is thus a faster evaporation of the liquid.

Mean pore size, here, is to mean in particular the ratio between four times the volume and the area of the pores, i.e. 4 V/A, as is stated in particular in the ISO Standard 15901.

As described above, the evaporator ceramic can be employed in particular for receiving low-viscosity liquids. Low-viscosity liquids are to mean in particular liquids having a viscosity of 45 mPas and lower.

The liquid can be any liquid. In particular it is possible to employ liquids containing medical substances.

It is preferred when the pore sizes of the pores of the pore structure, at least for the greatest part, lie within the mean pore size. This means in particular that maximally 10% of the pores have pore sizes that are larger than 4 times the mean pore size. This results in that pores having pore sizes above the mean pore size are reduced, preferentially not present. As a consequence, the effects of pores with pore sizes above the mean pore size on the overall behaviour of the evaporator ceramic and consequently the effects of droplets with larger volumes in these pores on the overall behaviour of the liquid received in the evaporator ceramic are negligible or at least reduced. Thus, it can be prevented in particular that the liquid drains from the evaporator ceramic. Further, this results in that the droplets received in the pores corresponding to the size distribution of the pores substantially have the same volumes. This results in a homogeneous distribution over the volume of the ceramic of the liquid received in the evaporator ceramic. In addition to this, the liquid can thus be more homogeneously and/or controllably evaporated.

Embodiments are considered advantageous, in which the mean pore size is between 0.1 μm and 25 μm, preferably between 0.15 μm and 10 μm, particularly preferably between 0.2 μm and 5 μm. There is thus an advantageous interaction between the capillary forces received in the pores, and the distribution of the liquid in the volume of the evaporator ceramic, which result in an improved receiving of the liquid in the evaporator ceramic and an improved evaporation of the liquid received in the evaporator ceramic.

Practically, the current path of the evaporator is such that the electric current upon electrical supply mandatorily flows along the current path. This means in particular that an electrical bypass of the blocking conductor is not possible and/or not provided.

The substance is such as evaporates by heating. Thus, the substance is evaporable.

Advantageously, the substance is liquid. In particular, the substance is a liquid. Both low-viscosity and also high-viscosity liquids, i.e. liquids with different viscosities, are conceivable.

Basically, the evaporator device can comprise a single blocking conductor.

It is also conceivable to provide the evaporator device with two or more such blocking conductors. It is preferred when the blocking conductors are identical. An abrupt increase of the electrical resistance when exceeding the operation end temperature, here, is to mean such an increase which exceeds a linear increase.

It is preferred when at least one of the at least one blocking conductors, upon exceeding of the operation end temperature, exhibits a potential increase of the electrical resistance. Particularly preferred are embodiments, in which at least one of the at least one blocking conductors is configured in such a manner that its electrical resistance on exceeding the operation end temperature starts to increase exponentially. Embodiments are considered advantageous, in which the electrical resistance of at least one of the at least one blocking conductors, advantageously of the respective blocking conductor rises, in the 50° C. following the operation end temperature, by at least a power of ten. Thus, the evaporation parameters can be particularly easily and effectively controlled.

Embodiments are considered advantageous, in which at least one of the at least one blocking conductors, advantageously the respective blocking conductor, is configured as a PTC thermistor, wherein the operation end temperature is between a starting temperature and an end temperature of the at least one PTC thermistor. PTC thermistors have a characteristic current curve, wherein the electrical resistance from the starting temperature abruptly rises by multiple powers of ten. Thus it is achieved in this manner that the blocking conductor does not or preferably marginally influence the electrical resistance of the total evaporator device, in the following also referred to as total resistance up to the starting temperature, and that the blocking conductor has an influence increasing the total resistance only when the starting temperature is reached. In other words, up to the reaching of the operation end temperature, the total resistance is thus dominated by the evaporator ceramic and upon reaching the operation end temperature by the at least one blocking conductor. As a consequence, the operation in the thermal operating range can take place with reduced energy expenditure and thus increased efficiency. At the same time, a precisely defined and reliable interruption or at least reduction of the electrical supply takes place when the operation end temperature is reached.

Basically, the operation end temperature can be anywhere between the starting temperature and the end temperature, advantageously between the starting temperature and the rated temperature of the PTC thermistor.

It is advantageous when the operation end temperature corresponds to the starting temperature of the PTC thermistor. Thus it is achieved in particular that for reaching the operation end temperature no increased energy expenditure, in particular no increased current consumption, is necessary. This results in an increased efficiency of the evaporator device. In addition, the evaporator device can thus be operated easily and for an increased operating duration with batteries, in particular rechargeable batteries. Further, this results in that the blocking conductor in the operating range does not generate any or preferably no heat. There is thus an improved control over the evaporation parameters.

The respective at least one blocking conductor can be basically arranged in any way in the current path provided it is heat-transmittingly connected to the evaporator ceramic.

It is conceivable, in particular, to arrange at least one of the at least one blocking conductors between the evaporator and one of the connections. This allows a simple and compact design of the evaporator device.

Basically, the heat-transmitting connection between the respective blocking conductor and the evaporator ceramic can be freely configured in the current path, provided it is heat-transmittingly connected to the evaporator ceramic.

It is conceivable in particular to arrange at least one of the at least one blocking conductors between the evaporator and one of the connections. This allows a simple and compact design of the evaporator device.

Basically, the heat-transmitting connection between the respective blocking conductor and the evaporator ceramic can be freely configured.

Particularly preferred are embodiments, in which at least one of the at least one blocking conductors, advantageously the respective blocking conductor, lies flat on the evaporator ceramic. In particular, one of the at least one blocking conductors can lie flat directly on the evaporator ceramic. This results in a simple and compact design of the evaporator device, wherein at the same time there is a simple and reliable heat transmission from the evaporator ceramic to the blocking conductor. At the same time it is thus possible to easily arrange the blocking conductor in the current path.

The evaporator ceramic is advantageously formed integrally and contiguously. Here it is preferred when on at least one outer side of the evaporator ceramic a blocking conductor is arranged. Thus, a compact and simple manufacture and design of the evaporator device is possible.

It is also conceivable to form the evaporator ceramic into or multiple parts. Thus, the evaporator ceramic can have two evaporator bodies that are separate from one another. Between at least two of the at least two evaporator bodies, a blocking conductor can be arranged.

Basically, the evaporator, besides the evaporator ceramic, can also comprise further constituent parts, which can serve in particular for conducting the electric current.

Preferred are embodiments, in which the evaporator consists of the evaporator ceramic, i.e. exclusively comprises the evaporator ceramic. This results in a simplified manufacture of the evaporator device and at the same time a precise and/or simple control over the evaporation parameters, such as for example the definition of the volume for receiving the substance to be evaporated and/or the generated heat.

Basically, the respective at least one blocking conductor can be manufactured from any material provided that, upon exceeding the operation end temperature, it exhibits an abruptly increasing electrical resistance.

It is conceivable, in particular that at least one of the at least one blocking conductors is a ceramic.

Practically, the at least one blocking conductor is dimensioned in such a manner that, compared with the evaporator ceramic, it makes up a smaller portion of the evaporator device in terms of volume. This allows in particular a more compact design of the evaporator device. In addition, the total volume provided for storing the substance is thus defined or at least dominated by the evaporator ceramic.

Preferred are embodiments, in which at least one of the at least one blocking conductors is formed as a layer. In particular, the at least one blocking conductor thus has a volume that is substantially reduced compared with the evaporator ceramic.

Basically, the respective layer can be freely configured. In particular, at least one of the at least one layer can be configured as a film, a coating and the like.

As described above it is preferred when the electrical total resistance of the evaporator device in the thermal operating range is dominated by the evaporator, in particular by the evaporator ceramic, and above the operating range, i.e. on exceeding the operation end temperature, by the blocking conductor.

This is preferably realised in such a manner that the electrical resistance of the blocking conductor in the operating range corresponds to maximally half of the electrical resistance of the evaporator, in particular of the evaporator ceramic.

The electrical resistance of the at least one blocking conductor is composed in particular of the specific resistance and the volume or the distance along the current path. Accordingly, a reduction of the electrical resistance of the blocking conductor in the operating range can be achieved by reducing the relative volume of the blocking conductor in the evaporator device.

The evaporator, in particular the evaporator ceramic, preferentially has an electrical resistance which up to the operation end temperature, in particular in comparison with the increase of the resistance of the blocking conductor from the operation end temperature, increases marginally. Preferably, the electrical resistance of the evaporator, in particular of the evaporator ceramic, follows a course that is temperature-dependent in the operating range in such a manner that the resistance maximally increases with the temperature by a power of ten.

Preferred are embodiments, in which the at least one blocking conductor has a volume, in the following also referred to as blocking volume, which amounts to maximally 1/10 (one tenth) of the volume of the evaporator, in particular of the evaporator ceramic, in the following also referred to as evaporator volume. The significantly higher evaporator volume in comparison with the blocking volume results in particular in that the substance to be evaporated is received and stored exclusively or at least predominantly in the evaporator ceramic. In other words, compared with the evaporator ceramic, the blocking conductor has no or at least a negligible storage function of the substance. Thus, an improved control over the evaporation parameters can be achieved. At the same time it is thus possible, as explained above, to achieve a lesser influence of the resistance of the at least one blocking conductor over the total resistance of the evaporator device in the operating range.

It is to be understood that the evaporator device can also comprise three or more electrical connections, wherein the current path leads between two of the connections and the evaporator ceramic.

Preferably, the evaporator device is employed in an inhaler in order to, upon electrical supply, evaporate the substance.

Preferably, the inhaler is a mobile and manually portable inhaler which can thus be carried along. The advantages of the evaporator device described above allow a compact design of the inhaler with reduced energy consumption and more precise control of the evaporation parameters at the same time.

As described above, the inhaler can be operated discontinuously and/or continuously. The inhaler is configured accordingly.

It is possible, in particular, to evaporate a predetermined dose of the substance in a controlled, in particular complete manner.

The inhaler, in particular the evaporator, can basically be employed for evaporating any substances. It is possible, in particular, to employ the inhaler for evaporating substances containing medically active ingredients. In particular, the defined and/or controllable dosing allow/s a correspondingly precise dosing of the active substance administered to a patient. It is likewise conceivable to employ the inhaler or the inhaler device as or in an electronic cigarette.

Advantageously, the inhaler, besides the evaporator device, comprises electronics for supplying the evaporator device, which is for example electrically connected to the connections of the evaporator device. The electronics can establish and disconnect an electrical connection between the connections and an electric energy source, preferentially a rechargeable battery of the inhaler. The electronics can thus include a control device for controlling the inhaler, which is equipped for controlling the inhaler.

Upon exceeding of the operation end temperature the inhaler, in particular the electronics, can, because of the interruption or at least reduction of the electrical supply of the evaporator ceramic provided by means of the at least one blocking conductor can be free of relevant control electronics and/or relevant sensorics.

It is to be understood that besides the evaporator device an inhaler having such an evaporator device is also included in the scope of this invention.

Further important features and advantages of the invention are obtained from the subclaims, from the drawings and from the associated figure description by way of the drawings.

It is to be understood that the features mentioned above and still to be explained in the following cannot only be used in the respective combination stated but also in other combinations or by themselves without leaving the scope of the present invention.

Preferred exemplary embodiments of the invention are shown in the drawings and are explained in more detail in the following description, wherein same reference numbers relate to same or similar or functionally same components.

### BRIEF DESCRIPTION OF THE DRAWINGS

It shows, in each case schematically

FIG. 1 shows an isometric view of an evaporator device,

FIG. 2 shows a greatly simplified representation in the manner of a circuit diagram of an inhaler with the evaporator device,

FIG. 3 shows an isometric view of the evaporator device with another exemplary embodiment.

### DETAILED DESCRIPTION

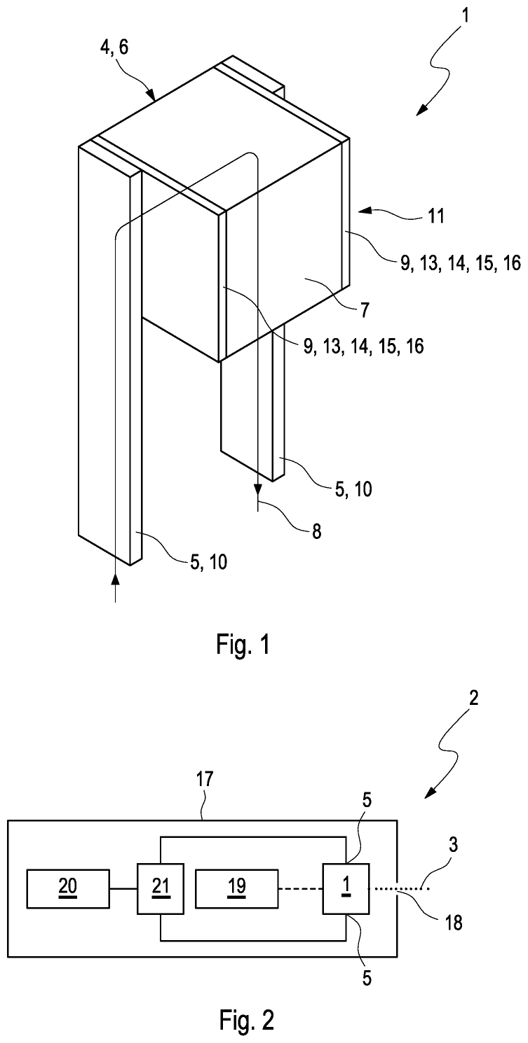

An evaporator device 1, such as is shown for example in the FIGS. 1 to 3, is employed in an inhaler 2, as it is exemplarily shown in FIG. 2 in a simplified manner. The inhaler 2 is a mobile manually portable inhaler 2, which in use is manually gripped and portable by a user who is now shown. The inhaler 2 is formed accordingly with respect to its dimensions.

The evaporator device 1 serves for evaporating a substance, in particular a predetermined dose of the substance (not shown). The substance is for example a substance which can contain a medically active ingredient, so that upon evaporation a vapour 3 (see FIG. 2) containing the active ingredient is dispensed, which is inhaled by a user.

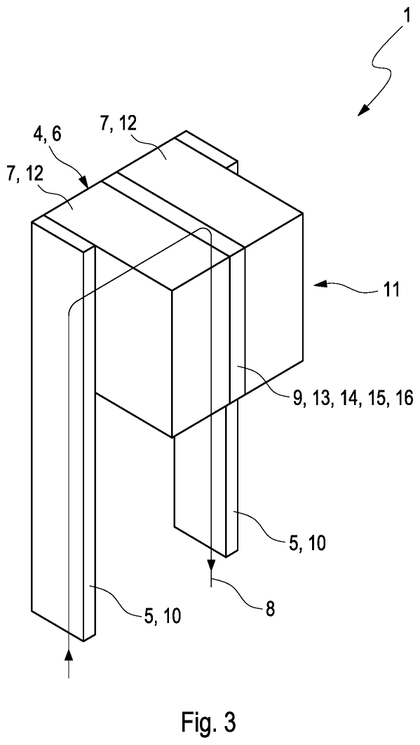

According to the FIGS. 1 and 3, the evaporator device 1 comprises an evaporator 4 and two electrical connections 5 for the electrical supply of the evaporator 4. The evaporator 4 serves both for receiving and storing the substance to be evaporated and also for generating heat for the purpose of evaporating the substance. For this purpose, the evaporator 4 comprises an electrically conductive ceramic 6, which in the following is also referred to as evaporator ceramic 6. In the shown exemplary embodiments, the evaporator 4 consists of the evaporator ceramic 6. For receiving and storing the substance to be evaporated, the evaporator ceramic 6 comprises a receiving structure 7, which preferably comprises pores (not shown), so that the substance to be evaporated is received in the pores. Advantageously, the receiving structure 7 consists of the pores, i.e. is a pore structure. In particular, the evaporator ceramic 6 can contain at least one metal oxide. For evaporating the substance, the evaporator ceramic 6 is operated in a thermal range, which in the following is also referred to as operating range. The operating range is delimited by a low temperature, in the following also referred to as operation starting temperature, and by a high temperature, in the following also referred to as operation end temperature. This means that the evaporation of the substance to be evaporated and received in the pores takes place in the operating range and thus between the operation starting temperature and the operation end temperature.

For generating heat, the evaporator 4 is electrically supplied by means of the connections 5, so that a path 8 of the electric current indicated in the FIGS. 1 and 3 leads between the connections 5 and through the evaporator 4. Upon electrical supply, the evaporator ceramic 6 generates, by means of its electrical resistance, heat for evaporating the substance. In this path 8, in the following also referred to as current path 8, at least one blocking conductor 9 is arranged in such a manner that the current path 8 mandatorily leads through the blocking conductor 9. In the shown exemplary embodiments, this is achieved in that the at least one blocking conductor 9 is arranged between the connections 5. The at least one blocking conductor 9 is heat-transmittingly connected to the evaporator ceramic 6. In the shown exemplary embodiments, the heat-transmitting connection of the at least one blocking conductor 9 with the evaporator ceramic 6 is realised by a flat arrangement of the blocking conductor 9 on the evaporator ceramic 6. In particular, the blocking conductor 9 lies directly against the evaporator ceramic 6. Thus, the temperature of the at least one blocking conductor 9 corresponds to the temperature of the evaporator ceramic 6. The at least one blocking conductor 9 is configured in such a manner that upon exceeding the operation end temperature it exhibits an abruptly increasing electrical resistance. Below the operation end temperature, the at least one blocking conductor 9 is thus electrically conductive, so that the evaporator ceramic 6 upon electrical supply is operated in the operating range, i.e. reaches temperatures up to the operation end temperature. The abrupt increase of the electrical resistance of the at least one blocking conductor 9 results in that the electric current flowing through the evaporator ceramic 6 upon exceeding of the operation end temperature is interrupted or substantially reduced, so that the abrupt increase of the electrical resistance of the at least one blocking conductor 9 defines or at least dominates the operation end temperature. Thus it is possible to operate the evaporator device 1 with controlled evaporation parameters. This allows in particular evaporating a predetermined quantity of the substance to be evaporated and thus a predetermined dose of the substance. For this purpose, separate control electronics (not shown) and/or separate sensorics (not shown) for example for determining the temperature of the evaporator ceramic is/are not necessary.

In the shown exemplary embodiments, the electrical connections 5 are ach formed as a circuit board 10, for example of a metal or a metal alloy. The evaporator 4 is arranged between the connections 5.

In the shown exemplary embodiments, the evaporator 4, in particular the evaporator ceramic 6, and the at least one blocking conductor 9 form a contiguous module 11, which is arranged between the connections 5. In the shown exemplary embodiments, the module 11 is of cuboid in shape. As is evident from the FIGS. 1 and 3, a volume portion of the evaporator ceramic 6 in the total volume of the module 11 is substantially larger than a volume portion of the at least one blocking conductor 9. In particular, the volume portion of the at least one blocking conductor 9, in the following also referred to as blocking volume, is maximally 1/10 of the volume portion of the evaporator ceramic 6, in the following also referred to as evaporator volume. This results in particular in that the volume for receiving the substance is determined or at least dominated by the evaporator ceramic 6. In addition, this results in that the at least one blocking conductor 9 in the operating range plays a negligible role in the electrical total resistance of the module 11 of the evaporator 4. In other words, the electrical total resistance of the evaporator 4 in the operating range is dominated by the evaporator ceramic 6, whereas above the operating range it is dominated by the at least one blocking conductor 9.

In the exemplary embodiment shown in FIG. 1, the evaporator ceramic 6 is formed contiguously and cuboid in shape, wherein between the respective outer side of the evaporator ceramic 6 facing one of the connections 5 and the associated connection 5 a blocking conductor 9 is arranged.

The exemplary embodiment shown in FIG. 3 differs from the exemplary embodiment shown in FIG. 1 in that the evaporator ceramic 6 is formed in two parts and thus comprises two evaporator bodies 12, which in the shown exemplary embodiment are each formed identical and cuboid in shape. Here, a single blocking conductor 9 is provided in the shown exemplary embodiment, which is arranged between the evaporator bodies 12.

In the shown exemplary embodiments, the respective blocking conductor 9 is formed as a, compared to the evaporator 6 or to the evaporator bodies 12, thin layer 13 and can therefore be also referred to as blocking layer 14.

The respective blocking layer 9 is preferentially a PTC thermistor 15, which from a starting temperature exhibits an abrupt electrical resistance increasing by multiple powers of ten. Here, the operation end temperature advantageously corresponds to a temperature between the starting temperature and an end temperature of the PTC thermistor 15, in particular the starting temperature of the PTC thermistor 15.

In particular, the PTC thermistor 15 is a ceramic 16 differing from the evaporator ceramic 6, which in the following is also referred to as blocking ceramic 16. Because of the low blocking volume of the blocking ceramic 16 compared with the evaporator volume of the evaporator ceramic 6, the total receiving capacity of the evaporator 4 is determined or at least dominated by the evaporator ceramic 6.

As is evident from FIG. 2, the inhaler 2 is a manually portable inhaler 2, in which the evaporator device 1 is received in a housing 17, which comprises an outlet opening 18 for letting out the vapour 3 produced by means of the evaporator 4. The inhaler 2 of the shown exemplary embodiment, further, comprises a preferentially refillable or replaceable container 19 for storing the substance to be evaporated, which, as indicated by a dashed line, is fluidically connected or connectible with the evaporator 4, in particular the evaporator ceramic 6.

Here, the evaporator device 1 and the container 19 can form a unit which is replaceably received in the inhaler 2.

Alternatively it is possible that the inhaler 19 is permanently received in the inhaler 2 and refillable. In this case, the evaporator device 1 can also be permanently received in the inhaler 2.

Alternatively it is possible that the container 19 is replaceable. In this case, the evaporator device 1 can also be permanently received in the inhaler 2.

The inhaler 2, further, comprises a rechargeable battery 20 for the electrical supply of the evaporator device 1, and electronics 21 electrically connected to the evaporator device 1. The electronics 21 is connected to the battery 20 in such a manner that for the purpose of the electrical supply of the evaporator device 1 it can establish and interrupt the electrical connection of the battery 20 to the evaporator device 1.

1. An evaporator device for evaporating a substance, comprising:
an evaporator configured to receive and evaporate the substance, the evaporator including an electrically conductive evaporator ceramic with a receiving structure such that during operation, the substance to be evaporated is received in the receiving structure;
two electrical connections for an electrical supply of the evaporator;
an electrical current path for the electrical supply of the evaporator extending through the two electrical connections and through the evaporator ceramic;
the evaporator ceramic configured to, during operation and upon electrical supply for evaporating the substance received in the receiving structure, homogeneously provide heat in a thermal operating range between an operation starting temperature and an operation end temperature;
at least one blocking conductor arranged in the current path and heat-transmittingly connected to the evaporator ceramic; and
wherein the at least one blocking conductor is configured such that, upon exceeding the operation end temperature, an electrical resistance of the at least one blocking conductor abruptly increases.

2. The evaporator device according to claim 1, wherein:
the at least one blocking conductor is configured as a PTC thermistor; and
the operation end temperature is between a starting temperature and an end temperature of the PTC thermistor.

3. The evaporator device according to claim 2, wherein the operation end temperature corresponds to the starting temperature of the PTC thermistor.

4. The evaporator device according to claim 1, wherein the at least one blocking conductor is arranged between the evaporator and one of the two electrical connections.

5. The evaporator device according to claim 1, wherein the at least one blocking conductor lies flat on the evaporator ceramic.

6. The evaporator device according to claim 1, wherein:
the evaporator ceramic includes two adjacent evaporator bodies; and
the at least one blocking conductor is arranged between the two adjacent evaporator bodies such that at least one evaporator body of the two adjacent evaporator bodies and the at least one blocking conductor are formed integrally.

7. The evaporator device according to claim 1, wherein the at least one blocking conductor is formed as a layer.

8. The evaporator device according to claim 1, wherein the receiving structure includes a plurality of pores for receiving the substance.

9. The evaporator device according to claim 1, wherein the electrical resistance of the at least one blocking conductor, in the thermal operating range, corresponds to half of an electrical resistance of the evaporator or less.

10. The evaporator device according to claim 9, wherein the electrical resistance of the evaporator, in the thermal operating range, increases by a power of ten or less.

11. The evaporator device according to claim 1, wherein a blocking volume of the at least one blocking conductor amounts to a tenth of an evaporator volume of the evaporator ceramic or less.

12. The evaporator device according to claim 1, wherein the evaporator consists of the evaporator ceramic.

13. An inhaler for evaporating a substance, comprising:
the evaporator device according to claim 1, and
a plurality of electronics electrically connected to the two electrical connections.

14. The evaporator device according to claim 1, wherein an electrical resistance of the evaporator, in the thermal operating range, increases by a power of ten.

15. The evaporator device according to claim 1, wherein the evaporator ceramic includes at least one metal oxide.

16. The evaporator device according to claim 1, wherein each of the two electrical connections is structured as a circuit board.

17. The evaporator device according to claim 1, wherein the evaporator ceramic and the at least one blocking conductor define a contiguous module that is cuboid in shape.

18. The evaporator device according to claim 1, wherein:
the at least one blocking conductor includes a first blocking conductor and a second blocking conductor;
the evaporator ceramic is arranged between the first blocking conductor and the second blocking conductor;
the first blocking conductor is arranged between the evaporator ceramic and a first electrical connection of the two electrical connections; and
the second blocking conductor is arranged between the evaporator ceramic and a second electrical connection of the two electrical connections.

19. The evaporator device according to claim 2, wherein:
the evaporator ceramic includes a first ceramic; and
the PTC thermistor includes a second ceramic that is different than the first ceramic.

20. A portable inhaler, comprising a substance and an evaporator for evaporating the substance, wherein:
the evaporator includes:
an electrically conductive and porous evaporator ceramic;
a plurality of electrical connections;
at least one blocking conductor heat-transmittingly connected to the evaporator ceramic; and
an electrical current path extending through the at least one blocking conductor and the evaporator ceramic between the plurality of electrical connections;
the substance is disposed in a plurality of pores of the evaporator ceramic at least during operation;
the evaporator ceramic is configured to, during operation, homogeneously provide heat in a thermal operating range between an operation starting temperature and an operation end temperature; and
upon exceeding the operation end temperature, an electrical resistance of the at least one blocking conductor abruptly increases such that the at least one blocking conductor at least one of reduces and interrupts an electrical supply of the evaporator ceramic.
